(19) United States
(12) Patent Application Publication
(10) Pub. No.: US 2022/0001211 A1
(43) Pub. Date: Jan. 6, 2022

(54) POSITIONING MECHANISM AND RADIOTHERAPY DEVICE

(71) Applicant: SHENZHEN OUR NEW MEDICAL TECHNOLOGIES DEVELOPMENT CO., LTD., Shenzhen City (CN)

(72) Inventors: Zhao GUO, Xi'an City, Shaanxi (CN); Ruirong ZHANG, Xi'an City, Shaanxi (CN)

(21) Appl. No.: 17/288,890

(22) PCT Filed: Sep. 5, 2019
(86) PCT No.: PCT/CN2019/104504

§ 371 Date: Apr. 26, 2021

(30) Foreign Application Priority Data
Oct. 26, 2018 (CN) 201811260080.9
Oct. 26, 2018 (CN) 201821764402.9

(51) Int. Cl. A61N 5/10

(52) U.S. Cl. CPC A61N 5/1081; A61N 5/1084

(57) ABSTRACT

A positioning mechanism includes: two lateral supports respectively connected to two sides of a treatment head; top supports connected to tops of the lateral supports; first positioning members disposed on the top supports; and second positioning members disposed on a top of a gantry. The first positioning members and the second positioning members are in one-to-one correspondence. A radiotherapy device is further provided.

### CROSS-REFERENCE TO RELATED APPLICATIONS

This application is a US national phase application of international application No. PCT/CN2019/104504, filed on Sep. 5, 2019, which claims priority to Chinese Patent Application No. 201811260080.9, filed on Oct. 26, 2018 and entitled “POSITIONING MECHANISM AND RADIOTHERAPY DEVICE,” and Chinese Patent Application No. 201821764402.9, filed on Oct. 26, 2018 and entitled “POSITIONING MECHANISM AND RADIOTHERAPY DEVICE,” the contents of each of which are herein incorporated by reference in their entirety.

### TECHNICAL FIELD

The present disclosure relates to the field of radiotherapy technologies, in particular to a positioning mechanism and a radiotherapy device.

### BACKGROUND

Gamma knives and medical linear accelerators are common radiotherapy devices used for treatment of tumors. For example, the gamma knife includes a gantry and a focused treatment head mounted on the gantry. Multiple radiation sources are mounted on the focused treatment head.

### SUMMARY

The present disclosure provides a positioning mechanism and a radiotherapy device.

Specifically, the following technical solutions are provided.

According to an aspect, a positioning mechanism is provided. The positioning mechanism includes: two lateral supports respectively connected to two sides of a treatment head;

top supports connected to tops of the lateral supports;

first positioning members disposed on the top supports; and

second positioning members disposed on a top of a gantry;

wherein the first positioning members and the second positioning members are in one-to-one correspondence.

In an optional embodiment, the positioning mechanism further includes an adjusting member; wherein

the adjusting member is movably mounted in or on the first positioning member, and provided with a mounting hole matching with the second positioning member; or

the adjusting member is movably mounted in or on the second positioning member, and provided with a mounting hole matching with the first positioning member.

In some embodiments, the first positioning member and the second positioning member are in plug connection, welded connection, bolt connection, or snap connection.

In an optional embodiment, the first positioning member is a positioning hole, and the second positioning member is a positioning pin; or

the first positioning member is a positioning pin, and the second positioning member is a positioning hole.

In an optional embodiment, the adjusting member is movably mounted on the positioning hole, and the positioning pin runs through the positioning hole and is secured into the mounting hole of the adjusting member.

In an optional embodiment, the first positioning member is an insertion hole, and the second positioning member is an insertion block; or

the first positioning member is an insertion block, and the second positioning member is an insertion hole.

In an optional embodiment, the adjusting member includes: an adjusting body, wherein the adjusting body is movably disposed inside the insertion hole and provided with the mounting hole; and

a fixing body, wherein the fixing body is connected to an end portion of the adjusting body and configured to secure the adjusting body into the insertion hole.

In an optional embodiment, the positioning mechanism further includes a reinforcing support, wherein two ends of the reinforcing support are respectively connected to the two lateral supports.

In an optional embodiment, the positioning mechanism further includes positioning supports, wherein the positioning supports are connected to the gantry and disposed between the lateral supports and the gantry, and thicknesses of the positioning supports are adjustable.

In an optional embodiment, the positioning mechanism further includes:

unloading supports, wherein the unloading supports are connected to the gantry and disposed between the lateral supports and the gantry, and thicknesses of the unloading supports are adjustable; and

the unloading supports are provided with rolling members, wherein the rolling members are configured to form rolling friction with the lateral supports

According to another aspect, a radiotherapy device is provided. The radiotherapy device includes: any of the positioning mechanisms mentioned above;

a treatment head, wherein the treatment head is connected to a lateral support of the positioning mechanism; and

a gantry, wherein the gantry is connected to a first positioning member on a top support by a second positioning member of the positioning mechanism.

### BRIEF DESCRIPTION OF THE DRAWINGS

For clearer descriptions of the technical solutions in the embodiments of the present disclosure, the following briefly introduces the accompanying drawings required for describing the embodiments. Apparently, the accompanying drawings in the following description show merely some embodiments of the present disclosure, and a person of ordinary skill in the art may still derive other drawings from these accompanying drawings without creative efforts.

FIG. 1 is a schematic structural diagram of a positioning mechanism according to an embodiment of the present disclosure;

FIG. 2 is a schematic structural diagram of a positioning mechanism equipped with an adjusting member according to an embodiment of the present disclosure;

FIG. 3 is a schematic structural diagram of another positioning mechanism according to an embodiment of the present disclosure, in which a positioning support and an unloading support are attached to but not installed on a lateral support;

FIG. 4 is a schematic structural diagram of a radiotherapy device according to an embodiment of the present disclosure;

FIG. 5 is a schematic structural diagram of a gantry;

FIG. 6 is a schematic structural diagram of a positioning support according to an embodiment of the present disclosure;

FIG. 7 is a schematic diagram of an operating relationship between a positioning support and a lateral support according to an embodiment of the present disclosure; and

FIG. 8 is a schematic structural diagram of an unloading support according to an embodiment of the present disclosure.

Reference numerals and denotations thereof:
1—lateral support; 101—connecting groove;
2—top support; 201—first positioning member; 202—second positioning member; 203—adjusting member;
3—positioning support; 301—first fixing block; 302—first thickness adjusting block;
4—unloading support; 401—rolling member; 4011—fixed body; 4012—ball;
402—second fixing block; 403—second thickness adjusting block;
5—reinforcing support; 6—via hole; 7—first driving member; 8—second driving member; 9—reinforcing block;
M—treatment head; N—gantry; P—mounting lateral plate.

### DETAILED DESCRIPTION

For clearer descriptions of the technical solutions and advantages of the present disclosure, embodiments of the present disclosure are described in detail hereinafter with reference to the accompanying drawings.

It should be noted that a treatment head of a radiotherapy device includes but is not limited to a therapy head and an imaging head. The treatment head is mounted on a gantry which is correspondingly provided with a mounting position to accommodate the treatment head. Generally, there are two mounting lateral plates at the mounting position of the gantry, wherein the lateral plates are configured to be connected to two sides of the treatment head (usually guide rails on two sides). It should be understood that the corresponding name of the treatment heads may be different depending on the specific radiotherapy devices. For example, a treatment head of a gamma knife is called a focused treatment head, and a treatment head of a linear accelerator is called an accelerator treatment head.

Since a plurality of radiation sources are mounted on the focused treatment head, in order to keep the plurality of radiation sources focusing on an isocenter, it is very necessary to ensure a mounting accuracy of the focused treatment head.

Currently, the focused treatment head is usually preliminarily mounted on the gantry, fitting spacers are disposed between guide rails on two sides of the focused treatment head and mounting lateral plates of the gantry on the same sides, and the position of the focused treatment head on the gantry is adjusted by selecting the fitting spacers with different thicknesses, so as to achieve a favorable mounting accuracy.

However, this process requires repeated mounting and dismounting of the heavy focused treatment head, which increases the mounting difficulty.

According to an embodiment of the present disclosure, a positioning mechanism facilitating mounting and positioning a treatment head is provided based on the above structures of the treatment head and the gantry. The structure of the positioning mechanism is briefly described hereinafter.

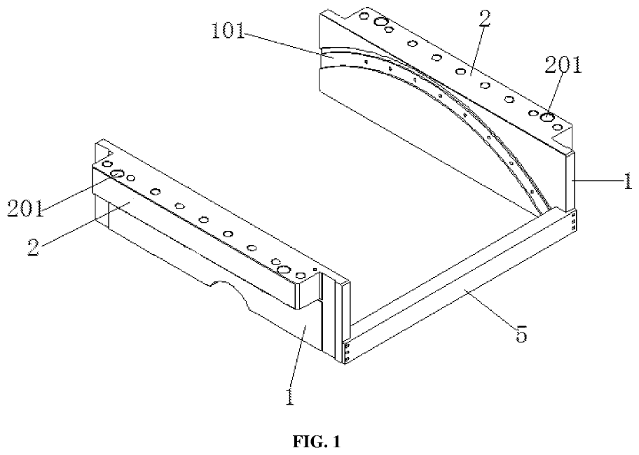

According to an aspect of embodiments of the present disclosure, a positioning mechanism is provided. As shown in FIGS. 1 to 5, the positioning mechanism includes: two lateral supports 1 respectively connected to two sides of a treatment head M of a radiotherapy device; top supports 2 connected to tops of the lateral supports 1; first positioning members 201 disposed on the top supports 2; and second positioning members 202 disposed on a top of a gantry N of the radiotherapy device. The first positioning members 201 and the second positioning members 202 are in one-to-one correspondence.

The positioning mechanism according to the embodiment of the present disclosure may be used for mounting and positioning the treatment head M in the radiotherapy device. In practice, the two sides of the treatment head M are connected to the lateral supports 1 of the positioning mechanism, and then an entirety thus formed is placed into a mounting position on the gantry N of the radiotherapy device for mounting. The second positioning members 202 are disposed at the top of the gantry N. First, the first positioning members 201 on the top supports 2 one-to-one correspond to the second positioning members 202, and the first positioning members and the second positioning members cooperate to achieve positioning of the treatment head M on the gantry N so as to improve the mounting accuracy. After the positioning is completed, the treatment head M is connected to the gantry N and secured onto the gantry N. It can be seen that the positioning mechanism according to the embodiment of the present disclosure can improve the mounting accuracy of the treatment head M, and the heavy treatment head M does not need to be repeatedly mounted and dismounted in the mounting process, which remarkably simplifies the mounting process and reduces the mounting difficulty.

There may be more than one first positioning member 201 on the same top support 2, e.g., two or three. It can be understood that the number of the second positioning members 202 equals the number of the first positioning members 201.

As mentioned above, the first positioning members 201 and the second positioning members 202 are in one-to-one correspondence, thereby realizing positioning of the treatment head M in the mounting process, wherein the positioning ways between the first positioning members 201 and the second positioning members 202 include but not limit to plug connection, bolt connection, welded connection, snap connection, or the like. For fast and effective connection, plug connection may be selected. The above connection modes will be described below.

For the bolt connection, both the first positioning members 201 and the second positioning members 202 may be bolt holes. In practice, the first positioning members 201 and the second positioning members 202 are connected in one-to-one correspondence, and bolts are used to connect with both of them in threaded connection, so as to achieve effective positioning.

For the welded connection, both the first positioning members 201 and the second positioning members 202 may be indicators. In practice, the first positioning members 201 and the second positioning members 202 are overlapped in one-to-one correspondence, and then the top support 2 is welded to the top of the gantry, so as to achieve effective positioning.

For the snap connection, the first positioning members 201 may be clamping grooves and the second positioning members 202 may be clamps. In practice, the second positioning members 202 may be clamped into the first positioning members 201, so as to achieve effective positioning.

The plug connection is simple, fast, and reliable. As an example, the first positioning members 201 are insertion holes and the second positioning members 202 are insertion blocks. Certainly, it does not exclude the case the first positioning members 201 are insertion blocks and the second positioning members 202 are insertion holes.

Generally, the structure of the insertion block matches with the insertion hole. For example, when the insertion hole is a circular hole, the insertion block is a cylindrical block; and when the insertion hole is a diamond hole, the insertion block is a diamond column.

As another example, the first positioning member 201 is a positioning hole and the second positioning member 202 is a positioning pin.

As another example, the first positioning member 201 is a positioning pin and the second positioning member 202 is a positioning hole.

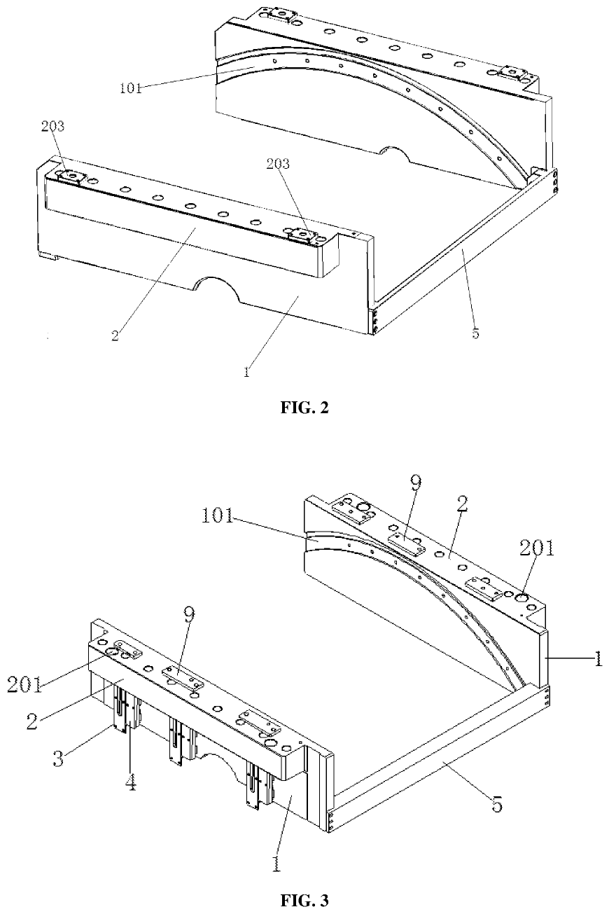

The outer contour of the insertion block (or the positioning pin) may exactly match with the inner contour of the insertion hole (or the positioning hole) to achieve a effect of stably positioning. The outer contour size of the insertion block (or the positioning pin) may also be smaller than the inner contour size of the insertion hole (or the positioning hole). For example, the radial width of the insertion hole (or the positioning hole) may be larger than that of the insertion block (or the positioning pin). In this case, as shown in FIG. 2, in an embodiment of the present disclosure, the top of the gantry N may be configured to support the top support 2. In other word, in practice, the top support 2 may be attached to a top wall of the gantry N.

Further, as shown in FIG. 2, the positioning mechanism according to the embodiment of the present disclosure further includes an adjusting member 203, wherein the adjusting member 203 is movably mounted in or on the first positioning member 201, and provided with a mounting hole matching with the second positioning member 202.

As another example, the adjusting member 203 is movably mounted in or on the second positioning member 202, and provided with a mounting hole matching with the first positioning member 201.

The adjusting member 203 is movably mounted in or on one of the positioning members, therefore the position of the treatment head M on the gantry N may be further appropriately adjusted, so as to achieve a higher mounting accuracy. The first positioning member 201 and the second positioning member 202 may be relatively fixed by forming the mounting hole on the adjusting member 203, wherein the mounting hole is used for matching with another positioning member.

As another example, when the first positioning member 201 and the second positioning member 202 work together by means of plug connection between the insertion hole and the insertion block, the adjusting member 203 may include an adjusting body, wherein the adjusting body is movably disposed in the insertion hole; and a fixing body, wherein the fixing body is connected to an end portion of the adjusting body. The adjusting body is provided with the mounting hole, and the fixing body is configured to secure the adjusting body into the insertion hole.

The position of the treatment head M on the gantry N can be finally adjusted by moving the adjusting body in the insertion hole. Upon determining the above positional relationship, the adjusting member 203 is fixed relative to the inside of the insertion hole through the fixing body, so as to ensure that the treatment head M is fixed relative to the gantry N.

The fixing body may be in a plate-shaped structure surrounding the end portion of the adjusting body. At this time, the fixing body may be secured to the top support by screws, so as to fix the adjusting member 203 relative to the insertion hole.

For example, when the first positioning member 201 and the second positioning member 202 are respectively a positioning hole and a positioning pin that match with each other, the adjusting member 203 is movably mounted on the positioning hole, and the positioning pin runs through the positioning hole and is secured into the mounting hole of the adjusting member 203. For example, as shown in FIG. 2, the adjusting member 203 may be platy, and may be mounted on a plate surface surrounding the positioning hole by screws, etc.

It can be known from the above that the two lateral supports 1 may be separated and relatively independent. For simplification of the structure of the positioning mechanism and improving its strength, as shown in FIG. 1, the positioning mechanism further includes a reinforcing support 5, wherein two ends of the reinforcing support 5 are respectively connected to the two lateral supports 1.

As another example, the reinforcing support 5 may be in a link structure, and there may be one or two reinforcing supports 5, wherein two ends of the reinforcing support 5 are respectively connected to the same side ends of the two lateral supports 1.

Further, the length of the reinforcing support 5 may be adjusted, such that the positioning mechanism may adapt to treatment heads M of various specifications.

As mentioned above, after the treatment head M is accurately positioned relative to the gantry N, the treatment head M and the gantry N are conventionally fastened and connected. Generally, a guide rail of the treatment head M and a mounting lateral plate P of the gantry N are connected by connecting screws. The second positioning member 202 may be disposed on the top of the mounting lateral plate P.

In view of that in practice, the lateral support 1 is clamped between the guide rail and the mounting lateral plate P, as shown in FIG. 6, the lateral support 1 is provided with a via hole 6 to allow the screws connecting the gantry N and the treatment head M to run.

The lateral supports 1 may be configured to be connected to the guide rails on the two sides of the treatment head M. According to the shapes of the guide rails, a connecting chute matching with the shape of the guide rails may be disposed on the lateral support 1. Generally, the guide rails on the two sides of the treatment head M are semi-circular arc. At this time, the semi-circular arc-shaped connecting groove 101 may be disposed on the lateral support 1, the guide rails may be clamped into the connecting groove 101 for preliminary positioning, and then, the only requirement is to connect by fasteners, e.g., screws. This arrangement is conducive to improving the connection strength and reducing the connection difficulty.

Further, as shown in FIG. 3, the positioning mechanism according to the embodiment of the present disclosure further includes a positioning support 3, wherein the positioning support 3 is connected to the gantry N and disposed between the lateral support 1 and the gantry N. The positioning support 3 is adjustable in thickness.

In practice, after the treatment head M is accurately positioned relative to the gantry N by the first positioning member 201 and the second positioning member 202, the treatment head M is preliminarily mounted on the gantry N. Since the positioning support 3 is connected to the gantry N, at this time the positioning support 3 is disposed between the lateral support 1 and the gantry N. By adjusting the thickness of the positioning support 3 until a mounting gap between the lateral support 1 and the gantry N is eliminated, i.e., the lateral support 1 and the positioning support 3 are closely attached to each other, the positioning support 3 may provide lateral support for the treatment head M, which effectively improves the mounting rigidity of the treatment head M. Then, the treatment head M may be subsequently connected and fastened to the gantry N.

Further, as shown in FIG. 3, the positioning mechanism may further include an unloading support 4, wherein the unloading support 4 is connected to the gantry N and disposed between the lateral support 1 and the gantry N. The unloading support 4 is adjustable in thickness and provided with a rolling member 401 which is configured to form rolling friction with the lateral support 1.

The unloading support 4 and the positioning support 3 may cooperate to dismount and re-mount the treatment head M. When the treatment head M is to be dismounted, the gantry N is rotated to dispose the treatment head M on one side of the gantry N (i.e., a central axis of the treatment head M extends in a horizontal direction). First, the preliminary positioning of the treatment head M relative to the gantry N is relieved. Then, the thickness of the positioning support 3 disposed between the gantry N and the treatment head M is adjusted until the mounting gap between the lateral support 1 and the gantry N is recovered, i.e., a gap is defined between the positioning support 3 and the lateral support 1 for the convenience of dismounting. After that, the thickness of the unloading support 4 disposed between the gantry N and the treatment head M is adjusted to eliminate the mounting gap between the lateral support 1 and the gantry N, and the unloading support 4 is configured to provide lateral support for the treatment head M, such that the treatment head M may be favorably positioned relative to the gantry N (this process can be understood as switching the positioning support 3 to the unloading support 4). Since the unloading support 4 is provided with the rolling member 401, which is configured to form rolling friction with the lateral support 1, based on the rolling friction, the treatment head M may be successfully pulled out of the gantry N by a treatment head dismounting tool (it should be understood that the treatment head M may be pulled out of the gantry N together with the lateral support 1 and the top support 2 connected thereto) after the fastening connection between the treatment head M and the gantry N is relieved. During remounting of the treatment head M, the mounting accuracy of the remounted treatment head M may be ensured only by returning to the original way and switching the unloading support 4 to the lateral support 1.

It can be seen that a mounting state of the treatment head M (i.e., the positioning support 3 acts) may be switched to a dismounting state (i.e., the unloading support 4 acts) by switching the positioning support 3 and the unloading support 4, and by the switching, the treatment head M may always maintain a favorable mounting accuracy when re-mounting after dismounted. Thus, on the premise of keeping the mounting accuracy, the treatment head M may be dismounted from the gantry N, and thus the treatment head M is operated as needed, such as component maintenance or source replacement, more convenient and feasible, which is of great significance for reducing the size and the cost of the radiotherapy device and effectively improving the product competitiveness.

The positioning support 3 and the unloading support 4 may be connected to the mounting lateral plates P of the gantry N. In this embodiment, the positioning support 3 can effectively improve the mounting rigidity of the treatment head M and provide stable lateral support for the treatment head M in use. For thickness adjustability of the positioning support 3, the applicable structure is briefly described hereinafter.

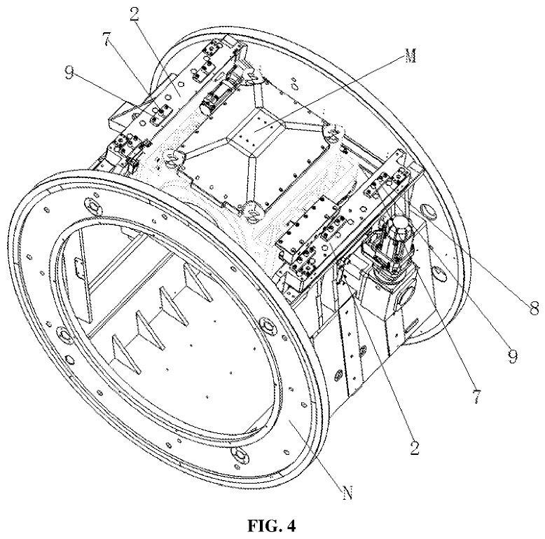

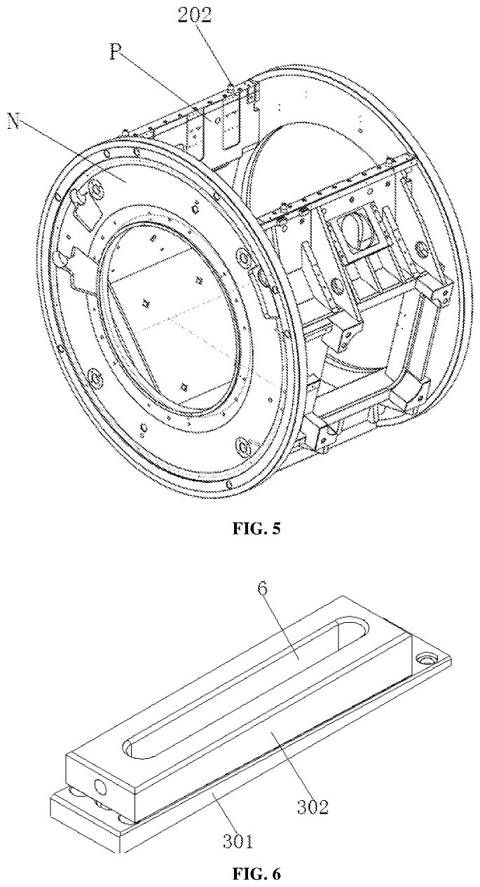

In an example embodiment, as shown in FIG. 6, the positioning support 3 includes a first fixing block 301 connected to the gantry N (e.g., a mounting lateral plate p of the gantry N in FIG. 5), and a first thickness adjusting block 302 slidable relative to the first fixing block 301. Furthermore, as shown in FIG. 4, the positioning mechanism further includes a first driving member 7 configured to drive the first thickness adjusting block 302 to bidirectionally slide.

In practice, the first fixing block 301 may be fixedly connected to the mounting lateral plate P of the gantry N, and the first thickness adjusting block 302 and the first fixing block 301 form a sliding pair. The first thickness adjusting block 302 can be driven to bidirectionally slide along the first fixing block 301 by the first driving member 7, such that the thickness of the positioning support 3 may be increased or reduced. Thus, the mounting gap between the lateral support 1 and the mounting lateral plate P of the gantry N can be eliminated or regained.

As for the sliding of the first thickness adjusting block 302 relative to the first fixing block 301, a sliding groove and a sliding rail may be disposed on their contact surfaces. For example, a sliding rail may be disposed on the first thickness adjusting block 302 and a sliding groove may be disposed on the first fixing block 301, such that the relative sliding may be achieved by cooperation between the sliding rail and the sliding groove. It should be understood that the sliding groove is longer than the sliding rail, such that the first thickness adjusting block 302 may bidirectionally slide along the first fixing block 301.

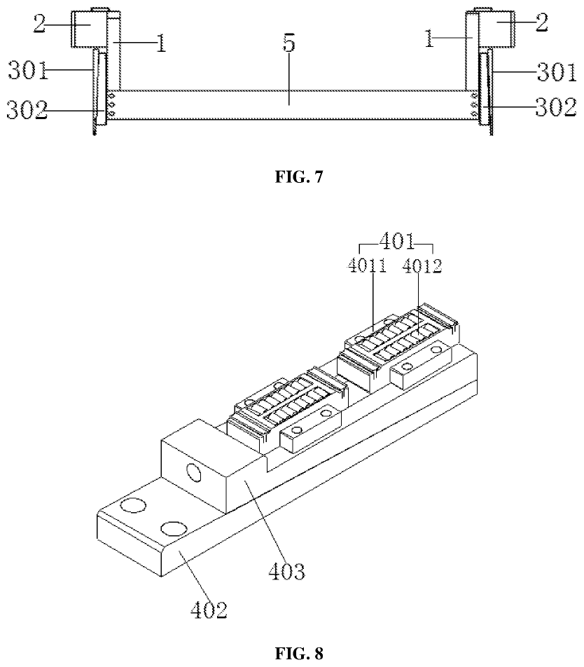

Considering that the first fixing block 301 is closely attached to the mounting lateral plate P of the gantry N, and the first thickness adjusting block 302 needs to be closely attached to the lateral support 1 (referring to FIG. 7), when the lateral support 1 is in a flat-plate-shaped structure, it is expected that the surface of the first thickness adjusting block 302 to which the lateral support is attached is also a plane. In view of this, for the purpose of thickness variation and ensuring that the first fixing block 301 and the first thickness adjusting block 302 are always closely attached to each other when the first thickness adjusting block 302 is bidirectionally sliding along the first fixing block 301, in this embodiment, as shown in FIG. 6, the first fixing block 301 and the first thickness adjusting block 302 are matched wedges, such that the first thickness adjusting block 302 may be tightly wedged with the lateral support 1 to form lateral support.

As another example, the first fixing block 301 may be a wedge with a rectangular profile, and its thickness may gradually decrease from top to bottom; and the first thickness adjusting block 302 may also be a wedge with a rectangular profile, and its thickness may gradually increase from top to bottom. That is, a fitting surface of the first fixing block 301 and the first thickness adjusting block 302 are a matched inclined surface.

As mentioned above, the first driving member 7 is configured to drive the first thickness adjusting block 302 to bidirectionally slide. As an example, the first driving member 7 and the first thickness adjusting block 302 may be in threaded connection, and thus by rotating the first driving member 7 in situ and changing a rotating direction, the first thickness adjusting block 302 may bidirectionally slide.

The first driving member 7 may be limited on the top support 2 to ensure the in-situ rotation. In an example embodiment, the first driving member 7 may be a bolt, which may run through the top support 2 and be in threaded connection to the first thickness adjusting block 302. Moreover, the first driving member may be always limited on the top support based on the limiting function of its head, so as to achieve the purpose of being rotatable but not axially movable. At the same time, when the first driving member is a bolt, the first thickness adjusting block 302 and the lateral support 1 can be kept in a wedged state all the time, thereby ensuring that the lateral support 1 and the mounting lateral plate P of the gantry N are always in a wedged state.

Further, a reinforcing block 9 may be disposed on the top support 2, such that the first driving member 7 may simultaneously run through the reinforcing block 9 and the top support 2 to be in threaded connection with the first thickness adjusting block 302. Thus, the strength at the joints may be improved. The reinforcing block 9 may be in a plate-shaped structure.

The unloading support 4 may be disposed on one or both of the two mounting lateral plates P of the gantry N at the same time. In view of that a favorable unloading effect may be achieved when the unloading support is only disposed on one side, in this embodiment, the unloading support 4 may only be disposed on the mounting lateral plate P on one side of the gantry N.

As mentioned above, the unloading support 4 is configured to laterally support the treatment head M in the process of dismounting the treatment head M. For thickness adjustability of the unloading support 4, the applicable structure is briefly described hereinafter.

In an example embodiment, as shown in FIG. 8, the unloading support 4 includes a second fixing block 402 connected to the gantry N (e.g., the mounting lateral plate P of the gantry in FIG. 5), and a second thickness adjusting block 403 slidable relative to the second fixing block 402. The rolling member 401 is disposed on the second thickness adjusting block 403.

Further, the positioning mechanism further includes a second driving member 8 configured to drive the second thickness adjusting block 403 to bidirectionally slide.

In practice, the second fixing block 402 may be fixedly connected to the mounting lateral plate P of the gantry N, and the second thickness adjusting block 403 and the second fixing block 402 form a sliding pair. The second driving member 8 may be used to drive the second thickness adjusting block 403 to bidirectionally slide along the second fixing block 402, such that the thickness of the unloading support 4 may be increased or reduced. Thus, the mounting gap between the lateral support 1 and the mounting lateral plate P of the gantry N can be eliminated or regained.

Reference may be made to the descriptions about the first thickness adjusting block 302 and the first fixing block 301 for the relative sliding manner of the second thickness adjusting block 403 to the second fixing block 402, which is not described repeatedly herein. In addition, the second fixing block 402 and the second thickness adjusting block 403 may also be matched wedges.

The second driving member 8 is in threaded connection to the second thickness adjusting block 403, so as to cause the second thickness adjusting block 403 bidirectionally slide. In an example embodiment, the second driving member 8 may also be a bolt.

Similarly, the second driving member 8 may also simultaneously run through the reinforcing block 9 and the top support 2 and be in threaded connection to the second thickness adjusting block 403. Thus, the strength at the joints may be improved. In this way, the unloading support 4 may be adjacently arranged to the positioning support 3 on the same side.

In this embodiment, the rolling member 401 is configured to form rolling friction with the lateral support 1. As an example, the rolling member 401 includes a fixed body 4011 secured onto the second thickness adjusting block 403 and a plurality of balls 4012 rotatably disposed on the fixed body 4011. The rolling friction may be formed between the balls 4012 and the lateral support 1. It should be understood that reference may be made to the structure of an abacus for the structure of the rolling member 401, which is not described in detail herein.

For optimization of the functions of the positioning support 3 and the unloading support 4, a plurality of positioning supports 3 and unloading supports 4 may be provided at the same time. In some embodiments, a plurality of positioning supports 3, e.g., 2, 3 (see FIG. 3) or 4, may be provided on the mounting lateral plate P on each side of the gantry N. Meanwhile, a plurality of unloading supports 4, e.g., 2, 3 (see FIG. 3) or 4, may be provided on the mounting lateral plate P on either side of the gantry N.

According to another aspect of the embodiments of the present disclosure, a radiotherapy device is further provided. The radiotherapy device includes: any one of the positioning mechanisms according to the embodiments of the present disclosure;

a treatment head, wherein the treatment head is connected to a lateral support; and

a gantry, wherein the gantry is connected to a first positioning member on a top support by a second positioning member of the positioning mechanism.

In view of the radiotherapy device, the following describes mounting and dismounting processes of the treatment head.

Mounting Process of Treatment Head:

First, a first fixing block of a positioning support of the positioning mechanism is connected to a mounting lateral plate of the gantry, and two lateral supports of the positioning mechanism are connected to guide rails on two sides of the treatment head. Then, the top support and the top of the mounting lateral plate of the gantry are positioned by the first positioning member and the second positioning member to achieve preliminary mounting of the treatment head on the gantry.

A first driving member may be configured to drive a first thickness adjusting block to move, such that the thickness of the positioning support may be increased until a mounting gap between the lateral support and the mounting lateral plate of the gantry is eliminated (i.e., the first thickness adjusting block is wedged tightly with the lateral support), and the positioning support is configured to provide lateral support for the focused treatment head, such that the focused treatment head may be accurately disposed relative to the gantry. Then, connecting screws are used to fasten and connect the guide rail of the focused treatment head to the mounting lateral plate of the gantry. In the above mounting process, a high mounting accuracy may be acquired, and multiple radiation sources on the focused treatment head can always focus on an isocenter.

Dismounting Process of Treatment Head:

A gantry bracket is rotated to drive the gantry to rotate, so as to position the treatment head on one side of the gantry (i.e., the axis of the treatment head extends in a horizontal direction). The preliminary positioning of the treatment head relative to the gantry can be relieved by removing pins at the top of the mounting lateral plate.

The first driving member can be used to drive the first thickness adjusting block to move in an opposite direction, such that the thickness of the positioning support is reduced until a mounting gap between the lateral support and the mounting lateral plate of the gantry is regained (i.e., the first thickness adjusting block is separated from the lateral support), which facilitates the disassembly. Then, the thickness of an unloading support between the mounting lateral plate of the gantry and the guide rail of the focused treatment head is adjusted. Specifically, a second thickness adjusting block is driven to slide along a second fixing block by a second driving member to increase the thickness of the unloading support, so as to eliminate the gap between the lateral support and the mounting lateral plate of the gantry (i.e., the second thickness adjusting block is wedged tightly with the lateral support). The unloading support is configured to provide lateral support for the focused treatment head, such that the focused treatment head is favorably positioned relative to the gantry (this process can be understood as switching the lateral support to the unloading support). Since the unloading support is provided with a rolling member, which is configured to form rolling friction with the lateral support, based on the rolling friction, the treatment head may be successfully pulled out of the gantry by a treatment head dismounting tool (it should be understood that the treatment head may be pulled out of the gantry together with the lateral support and the top support of the positioning mechanism) after the fastening connection between the treatment head and the gantry is relieved. During remounting of the treatment head, the mounting accuracy of the remounted treatment head may be ensured only by returning to the original way and switching the unloading support to the lateral support.

The treatment head dismounting tool and the operation processes of dismounting and mounting the treatment head are common in the art, which are explained by examples.

A base of the treatment head dismounting tool is secured to a foundation on a side of the gantry, and is reliably connected to the foundation by screws. The treatment head dismounting tool can linearly move in three orthogonal directions to adjust postures of the treatment head. The treatment head dismounting tool is connected to the gantry to keep the balance of the gantry in the dismounting process. A horizontal guide rail on the treatment head dismounting tool may extend below the guide rail of the treatment head inside the gantry.

In the dismounting process, by adjusting the height of the treatment head dismounting tool, the upper and horizontal guide rail on the treatment head dismounting tool may be closely attached to the guide rail of the treatment head to lift the treatment head with the unloading support. A horizontal lead screw on the treatment head dismounting tool is secured to the guide rail of the treatment head by an adapter plate. The treatment head may be moved out of the gantry along the horizontal direction by hand-cranking the horizontal lead screw.

Described above are only for the purpose of helping those skilled in the art to understand the technical solutions of the present disclosure, and are not intended to limit the present disclosure. Within the spirit and principles of the present disclosure, any modifications, equivalent substitutions, improvements, and the like are within the protection scope of the present disclosure.

1. A positioning mechanism, comprising:
two lateral supports respectively connected to two sides of a treatment head;
top supports connected to tops of the lateral supports;
first positioning members disposed on the top supports; and
second positioning members disposed on a top of a gantry;
wherein the first positioning members and the second positioning members are in one-to-one correspondence.

2. The positioning mechanism according to claim 1, further comprising an adjusting member, wherein
the adjusting member is movably mounted in or on the first positioning members, and provided with a mounting hole matching with the second positioning members.

3. The positioning mechanism according to claim 2, wherein the first positioning members and the second positioning members are in plug connection, welded connection, bolt connection, or snap connection.

4. The positioning mechanism according to claim 3, wherein
the first positioning members comprise a positioning hole, and the second positioning members comprise a positioning pin.

5. The positioning mechanism according to claim 4, wherein the adjusting member is movably mounted on the positioning hole, and the positioning pin runs through the positioning hole and is secured into the mounting hole of the adjusting member.

6. The positioning mechanism according to claim 3, wherein the first positioning members comprise an insertion hole, and the second positioning members comprise an insertion block.

7. The positioning mechanism according to claim 6, wherein the adjusting member comprises:
an adjusting body, wherein the adjusting body is movably disposed inside the insertion hole and provided with the mounting hole; and
a fixing body, wherein the fixing body is connected to an end portion of the adjusting body and configured to secure the adjusting body into the insertion hole.

8. The positioning mechanism according to claim 1, further comprising a reinforcing support, wherein two ends of the reinforcing support are respectively connected to the two lateral supports.

9. The positioning mechanism according to claim 1, further comprising positioning supports, wherein the positioning supports are connected to the gantry and disposed between the lateral supports and the gantry, and thicknesses of the positioning supports are adjustable.

10. The positioning mechanism according to claim 8, further comprising:
unloading supports, wherein the unloading supports are connected to the gantry and disposed between the lateral supports and the gantry, and thicknesses of the unloading supports are adjustable; and
the unloading supports are provided with rolling members, wherein the rolling members are configured to form rolling friction with the lateral supports.

11. A radiotherapy device, comprising:
a positioning mechanism, a treatment head, and a gantry, wherein the positioning mechanism comprises: two lateral supports respectively connected to two sides of the treatment head; top supports connected to tops of the lateral supports; first positioning members disposed on the top supports; and second positioning members disposed on a top of the gantry, wherein the first positioning members and the second positioning members are in one-to-one correspondence;
wherein the treatment head is connected to a lateral support of the positioning mechanism; and
wherein the gantry is connected to a first positioning member on a top support by a second positioning member of the positioning mechanism.

12. The positioning mechanism according to claim 1, further comprising an adjusting member, wherein the adjusting member is movably mounted in or on the second positioning members, and provided with a mounting hole matching with the first positioning members.

13. The positioning mechanism according to claim 3, wherein the first positioning members comprise a positioning pin, and the second positioning members comprise a positioning hole.

14. The positioning mechanism according to claim 3, wherein the first positioning members comprise an insertion block, and the second positioning members comprise an insertion hole.

15. The radiotherapy device according to claim 11, wherein the positioning mechanism further comprises an adjusting member, wherein the adjusting member is movably mounted in or on the first positioning member, and provided with a mounting hole matching with the second positioning member.

16. The radiotherapy device according to claim 15, wherein the first positioning member and the second positioning member are in plug connection, welded connection, bolt connection, or snap connection.

17. The radiotherapy device according to claim 16, wherein the first positioning member is a positioning hole, and the second positioning member is a positioning pin.

18. The radiotherapy device according to claim 17, wherein the adjusting member is movably mounted on the positioning hole, and the positioning pin runs through the positioning hole and is secured into the mounting hole of the adjusting member.

19. The radiotherapy device according to claim 16, wherein the first positioning member is an insertion hole, and the second positioning member is an insertion block.

20. The radiotherapy device according to claim 19, wherein the adjusting member comprises:
an adjusting body, wherein the adjusting body is movably disposed inside the insertion hole and provided with the mounting hole; and
a fixing body, wherein the fixing body is connected to an end portion of the adjusting body and configured to secure the adjusting body into the insertion hole.
